(19) United States
(12) Patent Application Publication
(10) Pub. No.: US 2022/0409669 A1
(43) Pub. Date: Dec. 29, 2022

(54) Nerve Xenografts and Related Methods

(71) Applicant: The General Hospital Corporation, Boston, MA (US)

(72) Inventors: Alexandre G. Lellouch, Harrison, NY (US); Curtis L. Cetrulo, Cohasset, MA (US)

(21) Appl. No.: 17/781,279

(22) PCT Filed: Dec. 2, 2020
(86) PCT No.: PCT/US2020/062895

§ 371 Date: May 31, 2022

Related U.S. Application Data
(60) Provisional application No. 62/942,419, filed on Dec. 2, 2019.

(51) Int. Cl. A61K 35/30; A61K 38/18; A61K 38/13; A61K 31/436; A61P 25/28

(52) U.S. Cl. CPC A61K 35/30; A61K 38/185; A61K 38/13; A61K 31/436; A61P 25/28

(57) ABSTRACT

Disclosed herein are nerve xenografts and methods of using such for repairing and/or protecting a nerve tissue in a human patient. The subject matter disclosed herein generally relates to nerve xenografts derived from genetically engineered source animals, and use of such nerve xenografts for repairing and/protecting nerve tissue in a human patient, e.g., for reconstruction of large peripheral nerve gaps, treatment of spinal cord injuries and ailments, and other therapies.

### CLAIM OF PRIORITY

This application claims the benefit of U.S. Provisional Patent Application No. 62/942,419, filed on Dec. 2, 2019, which is incorporated by reference herein in its entirety.

### FEDERALLY SPONSORED RESEARCH OR DEVELOPMENT

This invention was made with Government support under Grant No. OR160211 awarded by the Department of Defense. The Government has certain rights in the invention.

### FIELD OF THE INVENTION

The subject matter disclosed herein generally relates to nerve xenografts derived from genetically engineered source animals, and use of such nerve xenografts for repairing and/protecting nerve tissue in a human patient, e.g., for reconstruction of large peripheral nerve gaps, treatment of spinal cord injuries and ailments, and other therapies.

### BACKGROUND OF THE INVENTION

The urgent need for organs and other transplantable tissue has led to investigation into use of organs, cells, and/or tissue from non-human sources for xenotransplantation. Pigs have long been considered a potential non-human source of organs, cells, and/or tissue for use in human xenotransplantation given that their size and physiology are compatible with that of humans. Xenotransplantation from swine to humans, however, has significant roadblocks, not the least of which is hyperacute rejection where natural human antibodies target epitopes on the animal cells, causing rejection and failure of the transplanted organs, cells, and/or tissue.

### SUMMARY OF THE INVENTION

The present disclosure is based, at least in part, on the development of nerve xenografts for repairing and/or protecting nerve tissue in human patients, including improved immunosuppressive regimens that reduce the patient's exposure to an immunosuppressive agent, thereby reducing the patient's side effects to the immunosuppressive agent while allowing sufficient time for the endogenous nerve to regenerate and take the place of the nerve xenograft prior to its degradation by the patient's body. In such instances, the nerve xenograft functions as a conduit for nerve regeneration.

The present disclosure provides experimental data demonstrating the safety and effectiveness of xenogeneic nerve grafts from alpha-1,3-galactosyltransferase knockout (GalT-KO) swine for reconstruction of long segmental nerve gaps in Rhesus monkeys (Macaca mulatta) based on functional outcome, electrophysiologic data, and histological analysis. The total axonal regeneration and functional recovery after withdrawing immunosuppression is obtained after 6 months.

Accordingly, aspects of the present disclosure provide a nerve xenograft for implanting in a human patient, the nerve xenograft comprising nerve tissue derived from a genetically engineered non-human organism.

In some embodiments, the genetically engineered non-human organism has been genetically modified to lack alpha-1,3-galactose epitopes. In some embodiments, the genetically engineered non-human organism is a swine. In some embodiments, the nerve tissue is derived from the central nervous system of the genetically engineered non-human organism. In some embodiments, the nerve tissue is derived from the peripheral nervous system of the genetically engineered non-human organism.

In some embodiments, a nerve xenograft further comprises cells. In some embodiments, the cells comprise Schwann cells. In some embodiments, the cells are endogenous to the genetically engineered non-human organism or wherein the cells are endogenous to the human patient. In some embodiments, a nerve xenograft further comprises nerve growth factor (NGF).

In some embodiments, the genetically engineered non-human organism is an adult, a juvenile, or a fetus. In some embodiments, the nerve xenograft is cryogenically frozen. In some embodiments, the nerve xenograft is fresh.

Aspects of the present disclosure provide a method of repairing and/or protecting a nerve tissue in a human patient, the method comprising implanting a nerve xenograft described herein in a human patient in need thereof.

In some embodiments, the human patient is undergoing surgery that exposes nerve tissue. In some embodiments, the human patient has a nerve injury.

In some embodiments, the nerve injury comprises a peripheral nerve injury and/or a spinal cord injury. In some embodiments, the nerve injury is selected from the group consisting of a compressed nerve, a stretched nerve, a crushed nerve, a severed nerve, an inflamed nerve, a demyelinated nerve, and a nerve contusion.

In some embodiments, the nerve injury comprises a severed nerve, and wherein the severed nerve comprises a nerve gap. In some embodiments, the nerve gap is 3 to 7 centimeters in length. In some embodiments, the nerve gap is 3.5 to 4.5 centimeters in length.

In some embodiments, methods described herein further comprise administering to the human patient an immunosuppressive agent. In some embodiments, the immunosuppressive agent is a calcineurin inhibitor (CNI). In some embodiments, the calcineurin inhibitor is selected from the group consisting of cyclosporine, tacrolimus, and pimecrolimus. In some embodiments, the calcineurin inhibitor is administered at 0.05 to 5 mg/kg/day. In some embodiments, the calcineurin inhibitor is administered at 0.1 to 0.25 mg/kg/day. In some embodiments, the calcineurin inhibitor is administered at 0.15 mg/kg/day.

In some embodiments, the immunosuppressive agent is administered prior to implanting the nerve xenograft, concomitantly with implanting of the nerve xenograft, and/or after implanting of the nerve xenograft.

In some embodiments, the immunosuppressive agent is administered daily for 3 to 7 months. In some embodiments, the immunosuppressive agent is administered daily for 6 months.

In some embodiments, the nerve xenograft is implanted via surgery.

In some embodiments, methods described herein further comprise contacting the nerve xenograft with one or more biological molecules prior to implanting the nerve xenograft in the human patient.

### BRIEF DESCRIPTION OF THE DRAWINGS

FIGS. 1A-1B include bar graphs showing individual range of motion (ROM) analysis for animals in Group 1 (FIG. 1A; Group 1, 8.5 months on test, 8.5 months immunosuppression duration) and Group 2 (FIG. 1B; Group 2, 12 months on test, 6 months immunosuppression duration).

FIGS. 2A-2B includes bar graphs showing individual nerve conduction velocity (NCV) analysis for animals in Group 1 (FIG. 2A; Group 1, 8.5 months on test, 8.5 months immunosuppression duration) and Group 2 (FIG. 2B; Group 2, 12 months on test, 6 months immunosuppression duration).

FIGS. 3A-3C includes representative images of sections of the left proximal site of the xenograft from animal 3001 (Group 1, 8.5 months on test, 8.5 months immunosuppression duration). FIG. 3A includes a representative image of a neurofilament (NF) stained section showing both sides of longitudinal section of nerve containing proximal nerve (arrows), anastomosis (double-headed arrow) and graft region (arrowhead). FIG. 3B includes a representative image of a neurofilament (NF) stained section showing anastomosis site, which demonstrates neuroma (arrowheads). FIG. 3C includes a representative image of a hematoxylin and eosin (H&E) stained section showing lymphoid nodules (arrows) and hyalinized material (arrowhead), which likely represents residual graft material.

FIGS. 4A-4B includes representative images of sections of the left native graft site from animal 3001 (Group 1, 8.5 months on test, 8.5 months immunosuppression duration). FIG. 4A includes a representative image of a resected nerve stained for axon fibers. FIG. 4B includes a representative image of a resected nerve stained for myelin (exemplary myelinated nerves are indicated by arrows).

FIGS. 5A-5B includes representative images of sections of the left proximal graft site of the autograft from animal 3102 (Group 1, 8.5 months on test, 8.5 months immunosuppression duration) stained with axonal marker NF200 (FIG. 5A) or hematoxylin and eosin (FIG. 5B). FIG. 5A includes a representative image showing proximal nerve (arrow), anastomosis (double headed arrow), and proximal end of the graft (arrowhead) stained for axon fibers. FIG. 5B includes a representative image showing anastomosis site (double headed arrow) and graft (arrowheads).

FIG. 6 includes a representative image of a hematoxylin and eosin (H&E) stained section of the left distal site of the autograft from animal 3102 (Group 1, 8.5 months on test, 8.5 months immunosuppression duration). The distal nerve (arrow), anastomosis (double headed arrow), and distal end of graft (arrowheads) are shown.

FIG. 7 includes a representative image of a hematoxylin and eosin (H&E) stained section of the left proximal site of the xenograft from animal 4001 (Group 2, 12 months on test, 6 months immunosuppression duration). The proximal nerve, anastomosis, and proximal end of graft are shown. Arrows indicate residual sutures with foreign body response.

FIGS. 8A-8B includes representative images of hematoxylin and eosin (H&E) stained sections of the left distal site of the xenograft from animal 4001 (Group 2, 12 months on test, 6 months immunosuppression duration). FIG. 8A includes a representative image showing distal nerve, anastomosis, and proximal end of graft. Arrows indicate residual suture with foreign body response. Large arrowheads indicate lymphoid nodules in graft region. Small arrowheads indicate distal nerve. FIG. 8B includes a representative image showing lymphoid nodule (arrowheads), nerve fibers (large arrows), and minor edema in one of the nerve bundles (small arrows).

FIGS. 9-11 include representative images of a myelin-stained section of the left distal site of the xenograft from animal 4001 (Group 2, 12 months on test, 6 months immunosuppression duration). FIG. 9 shows distal nerve, anastomosis, and distal end of graft stained for myelin. Inset boxes A and B are shown at higher power in FIG. 10 and FIG. 11, respectively. Arrowheads indicate myelin formation.

FIG. 12 includes a representative image of a hematoxylin and eosin (H&E) stained section of the right proximal site of the autograft from animal 4102 (Group 2, 12 months on test, 6 months immunosuppression duration). The proximal nerve, anastomosis, and proximal end of graft are shown.

FIG. 13 includes a representative image of a hematoxylin and eosin (H&E) stained section of the right distal site of the autograft from animal 4102 (Group 2, 12 months on test, 6 months immunosuppression duration). The distal nerve, anastomosis, and distal end of graft are shown.

FIGS. 14-16 include representative images of a myelin stained section of the right distal site of the autograft from animal 4002 (Group 2, 12 months on test, 6 months immunosuppression duration). FIG. 14 shows distal nerve, anastomosis, and distal end of graft stained for myelin. Inset boxes A and B are shown at higher power in FIG. 15 and FIG. 16, respectively. Arrowheads indicate myelin formation.

The details of one or more embodiments of the invention are set forth in the description below. Other features or advantages of the present invention will be apparent from the following drawings and detailed description of several embodiments, and also from the appended claims.

### DETAILED DESCRIPTION

Aspects of the present disclosure provide nerve xenografts from a unique genetically modified non-human source animal whose tissues behave, in an immunologic sense, like human cadaver allograft tissue due to removal of alpha-1,3-galactose epitopes from such tissues. It was demonstrated, using nerve tissue from swine lacking alpha-1,3-galctose epitopes, that the nerve xenografts described herein performed as well as nerve autografts in reconstruction of nerve gaps in a Rhesus monkey model.

Xenotransplantation from swine to humans has significant roadblocks, not the least of which is hyperacute rejection where natural human antibodies target epitopes on the animal cells, causing rejection and failure of the transplanted organs, cells, and/or tissue. One cause of hyperacute rejection results from the expression of alpha-1,3-galactosyltransferase (“alpha-1,3-GT”) in porcine cells, which causes the synthesis of alpha-1,3-galactose epitopes. Except for humans, apes and Old-World monkeys, most mammals carry glycoproteins on their cell surfaces that contain galactose-alpha-1,3-galactose (see, e.g., Galili et al., “Man, apes, and Old-World monkeys differ from other mammals in the expression of a-galactosyl epitopes on nucleated cells,” J Biol. Chem. 263: 17755-17762 (1988). Humans, apes and Old-World monkeys have a naturally occurring anti-alpha gal antibody that is produced and binds to glycoproteins and glycolipids having galactose-alpha-1,3-galactose (see, e.g., Cooper et al., “Genetically engineered pigs,” Lancet 342:682-683 (1993). Accordingly, when natural type swine products are utilized in xenotransplantation, human antibodies will be invoked to confront the foreign alpha-1,3-galactose epitopes, and hyperacute rejection normally follows.

Peripheral nerve injury caused by ballistic trauma, automobile accidents, and other major trauma can be severe. Surgical autografts (i.e., nerve graft from the donor site) are currently utilized as the preferred standard of care, and are the most effective treatment for large nerve gaps. For example, the sural nerve in the human calf region can be utilized for autografts without causing severe lower limb disabilities. However, the availability of autografts is often insufficient especially when the patient has a large number of injuries, for example, today's wounded warrior who may have suffered unilateral or bilateral amputations. In cases where there is not sufficient donor autologous nerve, acellularized human allografts can be used. However, the decellularizing process removes the Schwann cells that support and promote nerve growth through the scaffold to reinnervate the target muscles. Ideally, a nerve graft should have viable components to promote axonal growth. To that end, transplanting large caliber, viable, human cadaver allograft nerves represents a more attractive strategy. One donor could provide ample donor nerve to address many defects, and large caliber single nerves such as the sciatic could be employed to reconstruct any size nerve defect.

Juvenile nerves (known to have far greater clinical regenerative capacity than adult nerves) would represent an even greater potential for improved functional outcomes. Juvenile cadaver donors, are, of course, logistically and ethically unrealistic and there is therefore a need for an alternative source of nerve grafts.

An advantage of using nerve xenografts described herein is that nerve tissue from source animals can be harvested along the spectrum of the animal's lifespan (from embryonic, to perinatal, to adult), with nerve tissue from the animal having different therapeutic characteristics depending on the age of the animal.

Accordingly, the present disclosure provides, in some aspects, nerve xenografts and methods of use thereof for repairing and/or protecting nerve tissue in a human patient. Methods for treating a nerve injury (e.g., a peripheral nerve injury and/or a spinal cord injury) in a human patient in need thereof are also within the scope of the present disclosure.

### I. Nerve Xenografts

Nerve xenografts disclosed herein comprise nerve tissue from a source animal such as those described herein. As used herein, nerve tissue refers to any tissue of the nervous system, e.g., the peripheral nervous system and/or the central nervous system. As used herein, a nerve xenograft refers to nerve tissue from a genetically engineered non-human source animal that is suitable for transplantation into a human patient.

The nerve xenografts described herein can comprise a variety of nerve tissue, e.g., nerve tissue derived from the peripheral nervous system (e.g., sciatic nerve) and/or the central nervous system (e.g., spinal cord) of a source animal. Nerve tissue can be obtained from any source animal such as those described herein.

The nerve xenograft described herein can comprise a variety of biological molecules that support transplantation of the nerve xenograft into a human patient, e.g., biological molecules involved in nerve growth, nerve repair, neuroprotection, and/or immunosuppression. Examples of biological molecules include, but are not limited to, cells, growth factors, and immunosuppressive agents.

Biological molecules can be obtained from any suitable source. In some embodiments, the nerve xenograft can comprise biological molecules that are endogenous and/or exogenous to the genetically engineered non-human organism (e.g., the swine). For example, the nerve xenograft can comprise nerve cells such as Schwann cells that are endogenous and/or exogenous to the genetically engineered non-human organism. In some embodiments, the nerve xenograft can comprise biological molecules that are endogenous and/or exogenous to the human patient. For example, the nerve xenograft can comprise nerve cells such as Schwann cells that are endogenous and/or exogenous to the human patient.

In some embodiments, the nerve xenografts described herein can comprise cells, e.g., cells that promote growth and regeneration of a nerve. For example, the nerve xenograft can comprise nerve cells such as glial cells (e.g., Schwann cells). Such cells can be obtained from a source animal or from another source (e.g., a cell culture). Cells can be naturally occurring cells (e.g., wild-type cells) or genetically modified cells. When a nerve xenograft comprises cells, the nerve xenograft can be referred to as cellularized. In other instances, the nerve xenograft can be treated (e.g., by chemical or mechanical methods) to remove cells. In such instances, the nerve xenograft can be referred to as acellularized. Methods and compositions provided herein encompass cellularized and acellularized nerve xenografts.

Examples of cells include, but are not limited to, glial cells (e.g., Schwann cells), progenitor cells, stem cells (e.g., mesenchymal stem cells), bone marrow derived cells (e.g., bone marrow stromal cells (BMSC)), mesenchymal stromal cells, dendritic cells, adipose cells, genetically modified cells (e.g., genetically modified Schwann cells), or a combination thereof. Such cells can be autologous, allogenic, isogenic, xenogeneic, or a combination thereof.

In some embodiments, the nerve xenograft described herein can comprise one or more growth factors. Examples of growth factors include, but are not limited to, nerve growth factor (NGF), brain derived nerve growth factor (BDNGF), vascular endothelial growth factor (VEGF), insulin growth factor (IGF-1), insulin-like nerve growth factor (IGF-2), epidermal growth factor (EGF), fibroblast growth factor (FGF), human growth factor (HGF), platelet-derived growth factor (PDGF), and neurotrophin-3 (NT-3).

Alternatively, or in addition to, a growth factor such as those described herein can be administered to a human patient via any suitable route, e.g., administered orally, parenterally, topically, and/or via an implantable depot (e.g., an injectable depot that releases an immunosuppressive agent). Parenteral administration includes, but is not limited to, subcutaneous, intracutaneous, intravenous, and intramuscular techniques. In some embodiments, the growth factor can be administered systemically (e.g., via intramuscular injection) or locally (e.g., via a nerve wrap or polymer).

In some embodiments, the nerve xenograft described herein can comprise one or more immunosuppressive agents. Examples of immunosuppressive agents include, but are not limited to corticosteroids, calcineurin inhibitors (e.g., cyclosporine, tacrolimus, and pimecrolimus), antimetabolites (e.g., azathioprine and mycophenolate mofetil), target of rapamycin inhibitors (e.g., sirolimus and everolimus), and antibodies (e.g., anti-lymphocyte antibodies and anti-cytokine receptor antibodies).

The nerve xenografts described herein can be a variety of shapes and sizes depending on various factors such as the properties of the nerve that is to be repaired and/or protected (e.g., nerve type, nerve diameter, nerve length), the type of nerve injury, and/or the condition of the human patient. For example, the nerve diameter of the nerve xenograft can be substantially similar to that of the injured nerve during gap reconstruction. In such examples, the nerve xenograft can be substantially tube shaped. In other examples, the nerve xenograft can be substantially flat for use as a sheath (e.g., the tube-shaped nerve xenograft can be split along the longitudinal axis and used as a flat sheath).

Nerve xenografts can comprise nerve tissue harvested using any method known in the art. For example, nerve tissue can be harvested by dissection of the nerve tissue from the source animal. Nerve tissue can be dissected free of adjoining tissue or dissected with portions of the surrounding tissue. Following harvesting, the nerve xenograft can be stored for later use. The nerve xenograft can be formulated for storage (e.g., formulated in storage media such as dimethilyosulfoxide) and cryopreserved for future use. Alternatively, the nerve xenograft can be fresh prior to use (e.g., unfrozen).

The therapeutic characteristics (e.g., immunogenicity and/or regenerative potential) of nerve tissue harvested from the source animals described herein (e.g., genetically modified swine) will vary depending on the age and stage of development of the animal. Accordingly, nerve xenografts described herein can comprise nerve tissue harvested from a source animal at any stage of its development (e.g., a source animal that is an adult, a juvenile, or a fetus). Accordingly, in some embodiments, the nerve xenograft comprises adult nerve tissue, juvenile nerve tissue, and/or fetal nerve tissue.

For example, it will be understood that nerve tissue derived from an adult source animal (e.g., an adult swine) can exhibit better immunogenicity but less regenerative capability than nerve tissue derived from a source animal that is perinatal or embryonic. It will also be understood that the nerve tissue derived from an adult source animal will tend to be larger than nerve tissue derived from a perinatal or embryonic source animal, and such nerve tissue will normally be less challenging to harvest (and therefore less costly) as compared to harvest from a perinatal or embryonic source animal. Accordingly, in some embodiments, the nerve xenograft comprises adult nerve tissue.

Similarly, it will be further understood that nerve tissue derived from a perinatal source animal (e.g., a perinatal swine) can exhibit better immunogenicity, but less regenerative capability than nerve tissue derived from a source animal that is embryonic. It will also be understood that nerve tissue derived from a perinatal source animal will be larger than nerve tissue derived from an embryonic source animal, and such nerve tissue will normally be less challenging to harvest (and therefore less costly) as compared to an embryonic source animal. Accordingly, in some embodiments, the nerve xenograft comprises perinatal nerve tissue.

Similarly, it will be further understood that nerve tissue derived from an embryonic source animal (e.g., an embryonic swine) can have less immunogenicity, but more regenerative capability than nerve tissue derived from a source animal that is adult or perinatal. It will also be understood that nerve tissue derived from an embryonic source animal will be smaller than nerve tissue derived from an adult or perinatal pig, and such nerve tissue will normally be more challenging to harvest (and therefore more costly) as compared to an adult or perinatal source animal. Accordingly, in some embodiments, the nerve xenograft comprises fetal nerve tissue.

### Genetically Engineered Non-Human Organisms

Nerve xenografts described herein comprise nerve tissue that is harvested from a genetically engineered non-human organism, which can also be referred to as a source animal. The genetically engineered non-human organism can be at any stage of its development when harvested for nerve tissue (e.g., the genetically engineered non-human organism is an adult, a juvenile, or a fetus).

Any suitable genetically engineered non-human organism can be used to prepare nerve xenografts described herein. Examples of genetically engineered non-human organisms include, but are not limited to, pigs, monkeys, sheep, goats, mice, cattle, deer, horses, dogs, cats, rats, mules, and other mammals. Genetically engineered non-human organisms encompasses non-mammalian animals including, but not limited to, birds, fish, reptiles, and amphibians.

In some embodiments, the genetically engineered non-human organism is a swine. As used herein, the terms “swine,” “pig” and “porcine” are generic terms referring to the same type of animal without regard to gender, size, or breed. In some embodiments, the genetically engineered non-human organism is a swine that has been genetically modified to lack alpha-1,3-galactose epitopes.

Genetically engineered non-human organisms described herein comprises at least one genetic modification. In some embodiments, the genetically engineered non-human organism comprises 1, 2, 3, 4, 5, or more genetic modifications. Genetically engineered non-human organisms can comprise any genetic modifications including, but not limited to, genetic modifications that reduce or eliminate immunogenicity, genetic modifications that promote or enhance nerve growth, and/or genetic modifications that promote or enhance nerve repair.

For example, a genetically engineered non-human organism can comprise a genetic modification that reduces or eliminates alpha-1,3-galactose epitopes, thereby reducing or eliminating immunogenicity of the harvested tissue. Any suitable genetic modification that reduces or eliminates alpha-1,3-galactose epitopes can be used (e.g., a genetic modification to galactose alpha-1,3 galactosyltransferase (GalT), which catalyzes the formation of the alpha-1,3-galactose epitopes on glycolipids, glycoproteins and polysaccharides).

Genetic modifications can be made by any method known in the art (e.g., CRISPR-Cas9 gene editing). See, e.g., Niu et al., “Inactivation of porcine endogenous retrovirus in pigs using CRISPR-Cas-9,” Science 357:1303-1307 (22 Sep. 2017).

A number of transgenic swine herds have been established that have reduced immunogenicity, and thus reduced likelihood of rejection, which can be used as a source of nerve xenografts for methods described herein. See, e.g., Denner J, “Xenotransplantation-Progress and Problems: A Review,” J Transplant Technol Res 4(2):133 (2014) (“Denner”)), the entire disclosure of which is incorporated herein by reference.

In some embodiments, the swine from which the subject nerve grafts are derived include “knockout” and/or “knock-in” swine such as are disclosed in U.S. Pat. No. 7,795,493 (“Phelps”), the entire disclosure of which is incorporated herein by reference. Such swine lack active alpha-1,3-galactosyl epitopes responsible for hyperacute rejection in humans upon transplantation. Multiple methods of production of knockout/knock-in swine are disclosed in Phelps including: the inactivation of one or both alleles of the alpha-1,3-GT gene by one or more point mutations (for example by a T-to-G point mutation at the second base of exon 9) and/or genetic targeting events as disclosed at col. 9, line 6 to col. 10, line 13; col. 21, line 53 to col 1.28, line 47; and col. 31, line 48 to col. 38, line 22.

Similarly, in other embodiments, the swine source animals can include the “knockout” and “knock-in” swine disclosed in U.S. Pat. No. 7,547,816 (“Day”), the entire disclosure of which is incorporated herein by reference. Such swine also lack active alpha-1,3-galactosyl epitopes responsible for hyper-acute rejection in humans upon transplantation. Multiple methods of production of knockout/knock-in swine are disclosed in Day including enucleating an oocyte, fusing the oocyte with a porcine cell having a non-functional alpha-1,3-GT gene, followed by implantation into a surrogate mother, as described more fully at col. 4, line 61 to col. 18, line 55. The creation of such swine through the described methods, and/or the utilization of such swine and progeny following creation, can be employed in the practice of the present invention, including, but not limited to, utilizing organs, tissue and/or cells derived from such swine.

Similarly, in other embodiments, the swine source animals can include the GGTA Null (“knockouts” and “knock-ins”) swine disclosed in U.S. Pat. No. 7,547,522 (“Hawley”), the entire disclosure of which is incorporated herein by reference. Such swine also lack active alpha-1,3-galactosyl epitopes responsible for hyper-acute rejection in humans upon transplantation. As disclosed in Hawley, production of knockout/knock-in swine includes utilizing homologous recombination techniques, and enucleating oocytes followed by fusion with a cell having a non-functional alpha-1,3-GT gene and implantation into a surrogate mother (as disclosed more fully at col. 6, line 1 to col. 14, line 31). The creation of such swine through the described methods, and/or the utilization of such swine and progeny following creation, can be employed in the practice of the present invention, including, but not limited to, utilizing organs, tissue and/or cells derived from such swine.

In yet other embodiments, the swine source animals can include the transgenic swine and swine that lack active alpha-1,3-galactosyl epitopes as disclosed in U.S. Pat. No. 9,883,939 (“Yamada”) at column 4, line 3 through line 54, the corresponding cited disclosure of which is incorporated by reference herein. The creation of such swine through the described methods, and/or the utilization of such swine and progeny following creation, can be employed in the practice of the present invention, including, but not limited to, utilizing organs, tissue and/or cells derived from such swine.

In yet other embodiments, swine source animals can include those disclosed in U.S. Pat. No. 8,106,251 (“Ayares”), U.S. Pat. No. 6,469,229 (“Sachs”), U.S. Pat. No. 7,141,716 (“Sachs”), each of which are incorporated herein by reference herein.

In some embodiments, the swine can originate from one or more highly inbred herds of pigs (whether genetically modified or not (i.e., wild-type)) with a co-efficient of inbreeding of 0.50 or greater. A higher coefficient of inbreeding allows the products derived from the source animals to have more consistent biological properties for use in pig-to-human xenotransplantation, and it will be understood that for product consistency, a coefficient of inbreeding of 0.80 or greater can be desirable in certain applications. Coefficients of inbreeding for animals are disclosed in Mezrich et al., “Histocompatible Miniature Swine: An Inbred Large—Animal Model,” Transplantation, 75(6):904-907 (2003), the entire disclosure of which is incorporated by reference herein. An example of a highly inbred herd of swine that can be utilized by this invention includes miniature swine descendant from the miniature swine disclosed in Sachs, et al., “Transplantation in Miniature Swine. I. Fixation of the Major Histocompatibility Complex,” Transplantation 22:559 (1976), which is a highly inbred line possessing good size matches particularly for organs eventually utilized for clinical transplantation.

It is therefore understood that multiple source animals, with an array of biological properties including, but not limited to, genome modification, and/or coefficients of inbreeding can be utilized to reduce immunogenicity and/or immunological rejection (e.g., acute, hyperacute, and chronic rejections) in humans resulting from xenotransplantation. It will be further understood that the listing of source animals set forth herein is not limiting, and the present invention encompasses any other type of source animal with one or more modifications (genetic or otherwise) that serve(s) to reduce immunogenicity and/or immunological rejection, singularly or in combination.

II. Use of Nerve Xenografts for Repairing and/or Protecting Nerve Tissue

In some aspects, provided herein are methods for repairing and/or protecting nerve tissue in a human patient using a nerve xenograft described herein. Such methods can comprise an immunosuppressive regimen, which comprises giving one or more doses of one or more immunosuppressive agents to the human patient.

To practice methods described herein, the nerve xenograft is implanted in a human patient in need thereof Implanting a nerve xenograft refers to contacting a nerve xenograft with a nerve tissue in a human patient for a sufficient time and in a sufficient manner to repair and/or protect the human patient's nerve tissue.

In some embodiments, implanting a nerve xenograft comprises attaching the nerve xenograft to a nerve tissue in need of repair and/or protection. For example, implanting a nerve xenograft can comprise attaching the nerve xenograft to each end of the severed nerve to reconstruct a nerve gap (e.g., reconstruction of a peripheral nerve gap). In another example, implanting a nerve xenograft can comprise wrapping the nerve xenograft around an injured nerve.

In some instances, the nerve xenograft can be degraded by the human patient's immune system, and endogenous regenerated nerve remains in place of the degraded nerve xenograft. Accordingly, the nerve xenograft can be referred to as a conduit.

The period of viability of the nerve xenograft after implantation can vary depending on factors such as the amount and/or duration of immunosuppressive agent(s) administered to the human patient, and/or the location of the nerve xenograft in the human patient. For example, the period of viability of the nerve xenograft after implantation can be as short as a few hours (e.g., 24 hours), to a few days, to as long as several months, or even as long as several years.

In some embodiments, implanting a nerve xenograft comprises covering or shielding a nerve tissue in need of repair and/or protection. For example, implanting a nerve xenograft can comprise covering a nerve tissue exposed during surgery (e.g., dura exposed during surgery).

The nerve xenograft can be implanted using any method known in the art, e.g., by surgically implanting the nerve xenograft in the human patient. Implanting a nerve xenograft can involve any suitable method or route that results in placement of the nerve xenograft at a desired site, such as at a site of a nerve injury. For example, implanting a nerve xenograft can include surgically attaching and/or wrapping the nerve xenograft to nerve tissue in a human patient. In other examples, implanting a nerve xenograft can include covering and/or shielding nerve tissue in a human patient (e.g., nerve tissue exposed during surgery). In such instances, the nerve xenograft can be removed from the patient prior to completion of surgery.

When applicable, methods described herein can comprise using a nerve xenograft comprising nerve tissue that is the same as, similar to, and/or different from the nerve being repaired or protected. For example, when repairing and/or protecting a nerve gap in a peripheral nerve of a human patient, the nerve xenograft can comprise nerve tissue derived from the peripheral nervous system of a source animal (e.g., a peripheral nerve from a GalT-KO swine). In another example, when repairing and/or protecting nerve tissue in the central nervous system of a human patient, the nerve xenograft can comprise nerve tissue derived from the peripheral nervous system of a source animal (e.g., a peripheral nerve from a GalT-KO swine).

Methods described herein can further comprise contacting a nerve xenograft (e.g., coating the nerve xenograft) with one or more biological molecules such as those described herein. The nerve xenograft can be contacted with one or more biological molecules at any suitable time prior to and/or during implanting the nerve xenograft in the human patient, e.g., prior to storage and/or during surgery.

### Immunosuppression

Any human patient suitable for the methods disclosed herein can receive one or more immunosuppressive agents to suppress or inhibit the human patient's immune system (e.g., immune cells such as T cells), thereby suppressing or inhibiting rejection of the nerve xenograft.

The immunosuppressive regimen described herein is administered under conditions that reduce the patient's side effects to the immunosuppressive agent while allowing sufficient time for the endogenous nerve to regenerate and take the place of the nerve xenograft prior to its degradation by the patient's body.

Immunosuppression refers to the partial or complete suppression of an immune response in a human patient. An “immunosuppressive agent” can be any molecule capable of reducing, depleting, or eliminating an immune response when administered to a human patient. In some embodiments, the immunosuppressive agent can be used to reduce immune rejection and/or enhance nerve regeneration.

Any suitable immunosuppressive agent can be used in the methods described herein. Examples of immunosuppressive agents include, but are not limited to, corticosteroids, calcineurin inhibitors, antimetabolites (e.g., azathioprine and mycophenolate mofetil), target of rapamycin inhibitors (e.g., sirolimus and everolimus), and antibodies (e.g., anti-lymphocyte antibodies and anti-cytokine receptor antibodies).

In some embodiments, the immunosuppressive agent is a calcineurin inhibitor. Calcineurin is a calcium and calmodulin dependent serine/threonine protein phosphatase that activates T cells via dephosporylation of cytoplasmic nuclear factor of activated T cell (NFATc). Dephosphorylated NFATc is translocated into the nucleus where it upregulates interleukin-2 (IL-2) expression, which then stimulates the growth and differentiation of T cells. Examples of calcineurin inhibitors include, but are not limited to, cyclosporine, tacrolimus, and pimecrolimus.

A human patient to be treated by methods described herein can receive a single immunosuppressive agent (e.g., tacrolimus) or a combination of immunosuppressive agents (e.g., tacrolimus in combination with azathioprine).

Methods disclosed herein encompass administration of an immunosuppressive agent in any suitable amount, e.g., an amount that provides partial or complete suppression of an immune response in a human patient.

For example, the calcineurin inhibitor (e.g., tacrolimus) can be administered at 0.05 to 5 mg/kg/day. In some embodiments, the calcineurin inhibitor (e.g., tacrolimus) can be administered at 0.1 to 5 mg/kg/day, 0.15 to 5 mg/kg/day, 0.25 to 5 mg/kg/day, 0.5 to 5 mg/kg/day, 0.75 to 5 mg/kg/day, 1 to 5 mg/kg/day, 2 to 5 mg/kg/day, 3 to 5 mg/kg/day, or 4 to 5 mg/kg/day. In some embodiments, the calcineurin inhibitor (e.g., tacrolimus) can be administered at 0.05 to 4 mg/kg/day, 0.05 to 3 mg/kg/day, 0.05 to 2 mg/kg/day, 0.05 to 1 mg/kg/day, 0.05 to 0.75 mg/kg/day, 0.05 to 0.5 mg/kg/day, 0.05 to 0.25 mg/kg/day, or 0.05 to 0.15 mg/kg/day. In some embodiments, the calcineurin inhibitor (e.g., tacrolimus) can be administered at 0.05, 0.1, 0.25, 0.5, 0.75, 1, 1.5, 2, 2.5, 3, 3.5, 4, 4.5, or 5 mg/kg/day.

A human patient can be administered any of the immunosuppressive agents for any suitable period such as disclosed herein. For example, the immunosuppressive agent can be administered daily to the human patient for 1 to 7 months (e.g., administered daily for 1, 2, 3, 4, 5, 6, or 7 months).

The immunosuppressive agent can be administered prior to implanting the nerve xenograft, concomitantly with implanting of the nerve xenograft, and/or after implanting of the nerve xenograft. In some embodiments, the immunosuppressive agent can be administered for 1 to 5 days (e.g., 1, 2, 3, 4, or 5 days) prior to implanting the nerve xenograft. In some embodiments, the immunosuppressive agent can be administered concomitantly with implanting the nerve xenograft (e.g., the immunosuppressive agent is administered on the same day as the nerve xenograft is implanted). In some embodiments, the immunosuppressive agent can be administered after implanting of the nerve xenograft for a suitable period (e.g., for 1 to 6 months after implanting of the nerve xenograft).

A human patient can be administered any of the immunosuppressive agents via any suitable route, e.g., administered orally, parenterally, topically, and/or via an implantable depot (e.g., an injectable depot that releases an immunosuppressive agent). The term parenterally includes, but is not limited to, subcutaneous, intracutaneous, intravenous, and intramuscular techniques. In some embodiments, the immunosuppressive agent can be administered systemically (e.g., via intramuscular injection) or locally (e.g., via a nerve wrap or polymer).

### Patients

A human patient can be any human subject for whom a nerve xenograft is desired. A human patient can be of any age. In some embodiments, the human patient is an adult (e.g., a person who is at least 18 years old). In some embodiments, the human patient is a child.

A human patient to be treated by the methods described herein can be a human patient having a nerve injury. A nerve injury refers to a nerve comprising any type of damage, dysfunction, or degeneration. Any nerve injury can be treated according to methods described herein. A nerve injury to be treated according to methods described herein can be caused by a variety of factors including, but not limited to, disease, trauma, surgery, and combinations thereof.

A nerve injury can include a partial or complete interruption of continuity in a nerve, which results in the nerve having a partial or complete interruption of conduction. A nerve injury can include an intact nerve that is injured and/or a nerve that is severed. Examples of a nerve injury include, but are not limited to, a nerve gap, a compressed nerve, a stretched nerve, a crushed nerve, a severed nerve, an inflamed nerve, a demyelinated nerve, a nerve contusion, and a combination thereof. Accordingly, a nerve injury can comprise compression, inflammation, demyelination, and/or bruising.

A served nerve can comprise a nerve gap of any length. In some embodiments, the severed nerve comprises a nerve gap of 3 to 7 centimeters in length. In some embodiments, the severed nerve comprises a nerve gap of 4 to 7 centimeters in length, 5 to 7 centimeters in length, 6 to 7 centimeters in length, 3 to 6 centimeters in length, 3 to 5 centimeters in length, or 3 to 4 centimeters in length. In some embodiments, the severed nerve comprises a nerve gap of about 3 centimeters in length, about 4 centimeters in length, about 5 centimeters in length, about 6 centimeters in length, or about 7 centimeters in length. In some embodiments, the severed nerve comprises a nerve gap of 3.5 to 4.5 centimeters in length.

A nerve injury can alter any portion of the nerve structure including, but not limited to, epineural sheath, epineurium, perineurium, fascicles, endoneurium, myelin, neurons, nerve fibers, and combinations thereof.

A human patient can have an injury to any nerve in the body, e.g., a peripheral nerve injury and/or a spinal cord injury. Accordingly, in some embodiments, methods described herein comprise treating a human patient having a peripheral nerve injury. In some embodiments, methods described herein comprise treating a human patient having a spinal cord injury. A human patient who needs a nerve xenograft can be identified by routine medical examination, e.g., electromyography (EMG) and/or magnetic resonance imaging (MRI).

Following implantation of a nerve xenograft, a human patient can be monitored for acute toxicities such as graft versus host disease (GvHD) and cytokine release syndrome (CRS). When a human patient exhibits one or more symptoms of acute toxicity, the human patient can be subjected to toxicity management. Treatments for patients exhibiting one or more symptoms of acute toxicity are known in the art. For example, a human patient exhibiting a symptom of CRS (e.g., respiratory and/or neurological abnormalities) can be administered an anti-cytokine therapy.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described can be more fully understood, the following examples are set forth. The examples described in this application are offered to illustrate the methods and compositions provided herein and are not to be construed in any way as limiting their scope.

### Example 1: Reconstruction of Large Nerve Gaps Using GalT-KO Porcine Nerve Xenografts in Rhesus Monkeys

This example describes studies performed to evaluate the safety and effectiveness of xenogenic nerve grafts from alpha-1,3-galactosyltransferase knockout (GalT-KO) swine for reconstruction of long segmental nerve gaps in Rhesus monkeys (Macaca mulatta) based on functional outcome, electrophysiologic data, and histological analysis. The studies described herein also evaluated the effect on functional recovery of withdrawing immunosuppression after axonal regeneration through the graft was expected to be complete (after 6 months).

A total of ten rhesus monkeys were enrolled in the studies described herein. Immunosuppression therapy was initiated 5-10 days before surgical procedures via intramuscular injection of tacrolimus. On Day 0, an approximately 4 cm segment of the parent radial nerve was surgically removed to create a nerve defect in both the left and right arms. In one arm, a xenogenic nerve graft (also referred to as xenograft or Xeno-Nerve) from a GalT-KO swine, prepared fresh for implant or frozen and thawed, was attached proximally and distally using nylon sutures at each neurorrhaphy site. On the opposite side, the nerve section removed to create the defect was rotated 180 degrees and re-attached in the same manner as the xenograft. The native nerve graft collected from the parent radial nerve on the xenograft side was saved stored at room temperature in 10% neutral buffered formalin (NBF). Animals were then survived for 8.5 (Group 1) or 12 months (Group 2), during which time immunosuppression therapy continued for 6 months (Group 2) or until necropsy (Group 1).

Animals were treated identically for the first 6 months, including monthly assessments of functional recovery. For Group 1, following the 6-month functional recovery testing, electrophysiological nerve studies were performed, and then the grafted nerve segment (including 1 cm of proximal and distal radial nerve stumps) was excised for histologic analysis of fiber count, number, myelinated proportion, and identification of any immune cell infiltration.

For Group 2, immunosuppression was withdrawn following the 6-month functional recovery testing, and functional recovery was monitored at 2-week intervals for an additional 3 months. At 9 months, Group 2 was assessed by electrophysiological nerve studies, and then the grafted nerve was excised for histologic analysis in the same manner as Group 1.

A study summary is provided in Table 1.

TABLE 1
Study Summary.

| | Number | | | |
|---|---|---|---|---|
| | of | Initial Wound Creation and | | Necropsy |
| Group | Animals | Xenograft Placement (Day 0) | Immunosuppression | (Months) |
| 1 | 5 | An approximate 4 cm radial | Tacrolimus at 0.15 mg/kg/day | 8.5 |
| | | nerve defect was created | (target dose) until necropsy | |
| 2 | 5 | surgically in each forearm | Tacrolimus at 0.15 mg/kg/day | 12 |
| | | (bilateral) | (target dose) until 6 months | |
| | | Side 1: received porcine | (no tacrolimus for final 6 months) | |
| | | nerve xenograft to repair | | |
| | | the defect (fresh or frozen)* | | |
| | | Side 2: received autograft | | |
| | | nerve to repair the defect | | |
| | | (removed nerve was flipped | | |
| | | and replaced at the same site) | | |

*Overall, five animals received previously frozen xenograft and five received fresh xenograft.

### Example 2: Nerve Xenografts Restored Range of Motion (ROM) in a Similar Manner to Nerve Autografts in Rhesus Monkeys

The objective of this study was to assess the ability of nerve xenografts to restore range of motion (ROM) when used for reconstruction of long segmental nerve gaps in Rhesus monkeys.

Wrist extension functional assessments were performed using multiple mechanisms appropriate for each animal, including chair and/or cageside observation of active and passive wrist angle, flexion. Cageside evaluations included retrieval of food treats in physical locations that would require wrist angle extension to obtain them. Chaired observations were performed using food treats or mechanical stimulation to encourage wrist angle extension from neutral (in line with the forearm, 0°). Angle data were converted to a range-of-motion (ROM) score by assigning a numerical value from 1-4 for every 30° of wrist flexion. This scoring method was chosen to balance the limited figure of merit for assessing angular extension. The precision of the ROM measurement is inherently limited to gross observations, but do allow for more detailed results than binary-only outcomes. Thus, the ROM score was defined as: angles less than 31° (Score 1), 31°-60° (Score 2), 61° to 90° (Score 3), and >91° (Score 4), respectively.

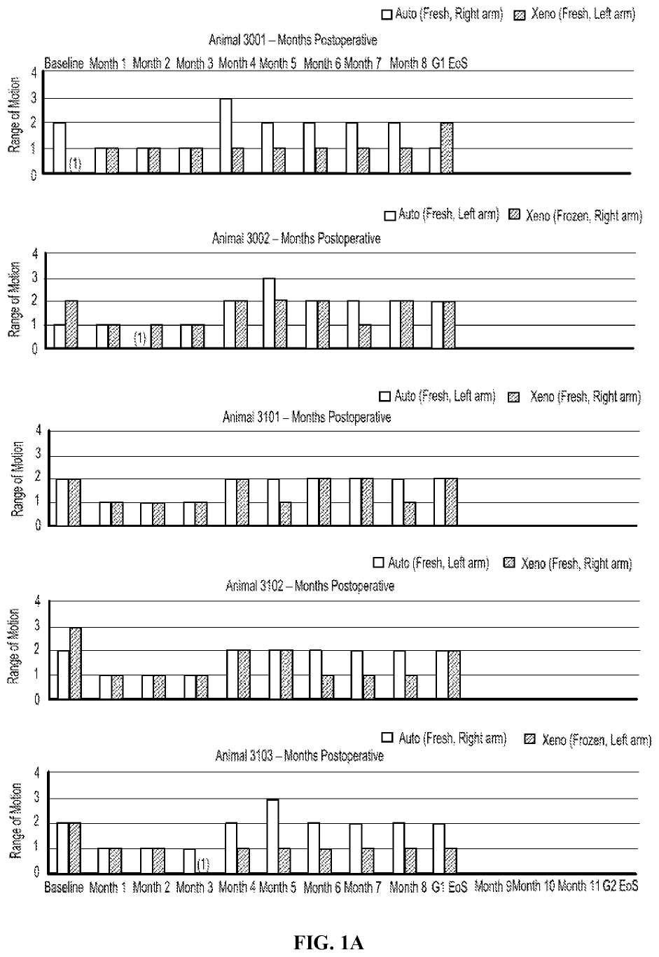

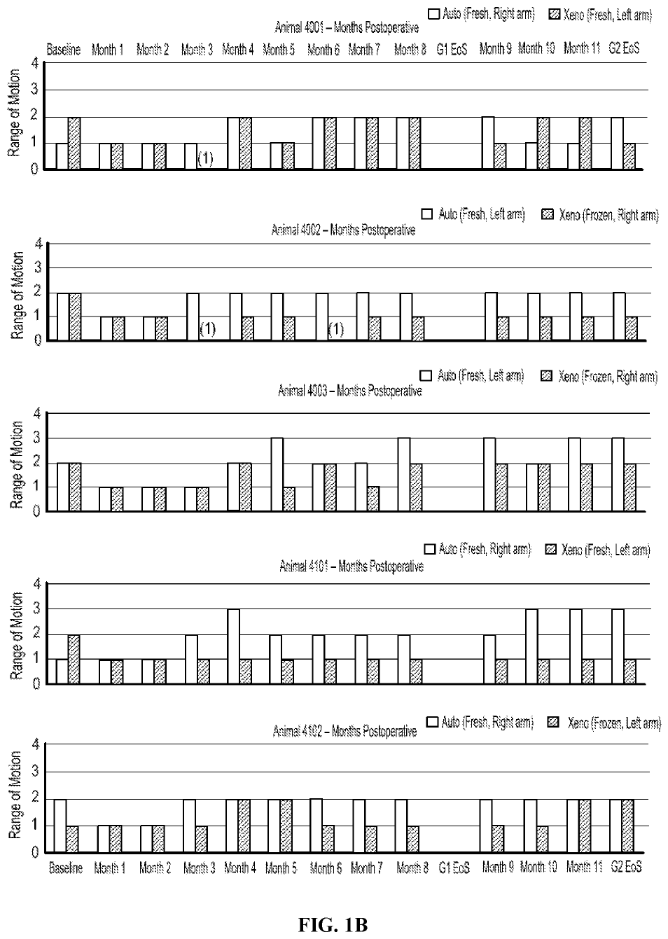

Surgical nerve injury was associated with immediate and severe decreases in ROM that persisted for approximately 3 months in most animals in both treatment groups regardless of graft type. By Month 4 postoperative, animals began demonstrating functional recovery, which subsequently began approaching baseline levels (Group 1: FIG. 1A; and Group 2: FIG. 1B) with time. By the end of the 12-month study, all 10 autografted repairs and 7 xenografted repairs showed ROM equal to baseline values. In some cases, Xeno-Nerve-grafted limbs exhibited relatively slower, more gradual recovery during the duration of the study. However, by the end of the study at Month 12, limbs transplanted with Xeno-Nerve showed equivalent maximal recovery indistinguishable from the maximal recovery in autografted limbs in the majority of subjects. Functionally, the wrist extension data demonstrates similar results in Xeno-Nerve-grafted and autografted limbs for most animals, with the most pronounced range of motion recovery realized by month four. Animals in both groups generally maintained the 4-month level of recovered extension through the remainder of the study. Variation in the month-over-month data appears due to the difficulty and imprecision of the ROM testing schema.

Xeno-Nerve was as effective at restoring substantial ROM comparable to autografts for most animals by the end of the study, only requiring slightly longer recovery durations in a few cases. One of 5 animals in Group 1 that had not recovered similar ROM on the Xeno-Nerve side as the autograft side was still demonstrating monthly improvements at the last data collection, 8.5 months post graft. At this 8.5-month time, nerve conduction values indicated axonal regeneration had occurred and delayed but eventual regain-of-function can have been observed with extended recovery time, as suggested by animals with similar trends in Group 2.

Taken together, these results demonstrate that nerve xenografts were as effective as autografts in restoring ROM when used for reconstruction of long segmental nerve gaps in a Rhesus monkey model.

### Example 3: Nerve Xenografts Restored Nerve Conduction in a Similar Manner to Nerve Autografts in Rhesus Monkeys

Nerve injury/regeneration was evaluated non-invasively on anesthetized animals by testing the ability of the nerve to conduct an electrical impulse. The nerve was stimulated at one or more sites along its course, and the electrical response of the nerve was recorded. Locations for stimulation and recording were chosen relative to anatomical landmarks (e.g., elbow, spiral groove, axilla). For sensory branches, the recording electrode was positioned directly over the distal branches of the nerve. For radial motor branches, the recording electrode was positioned over the belly of the Extensor Digitorum Communis muscle (EDC; innervated by the radial nerve). The recording location was at the junction of the upper third and middle third of the forearm. The Nerve Conduction Velocity (NCV) was expected to vary across the length of the nerve with progressive recovery, and therefore stimulation was performed at four locations proximal and distal from the graft (e.g., lateral from the ulna or biceps tendon in the antecubital fossa; at the spiral groove; across the graft; and at the axilla between the coracobrachialis and the long head of the biceps).

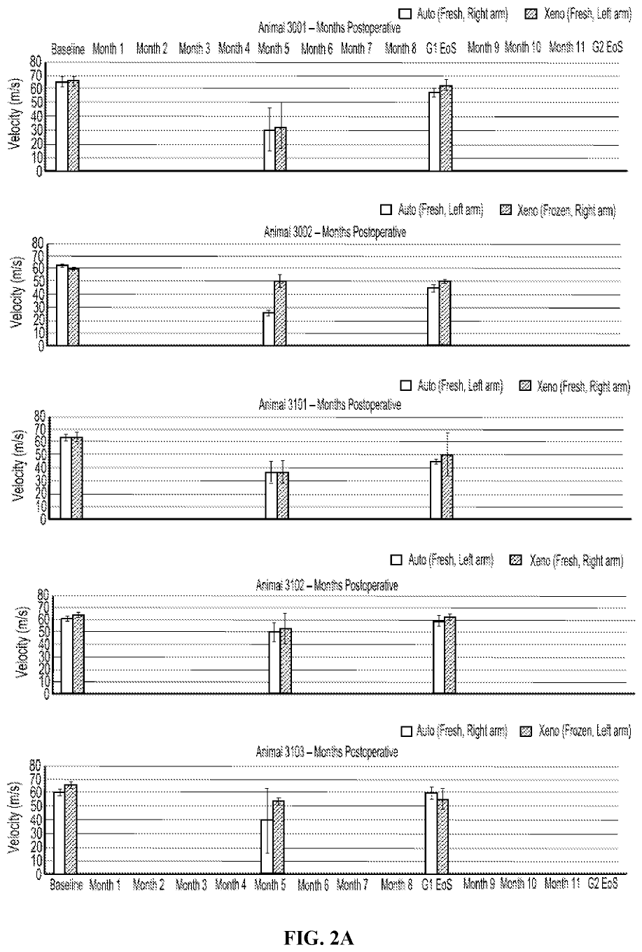

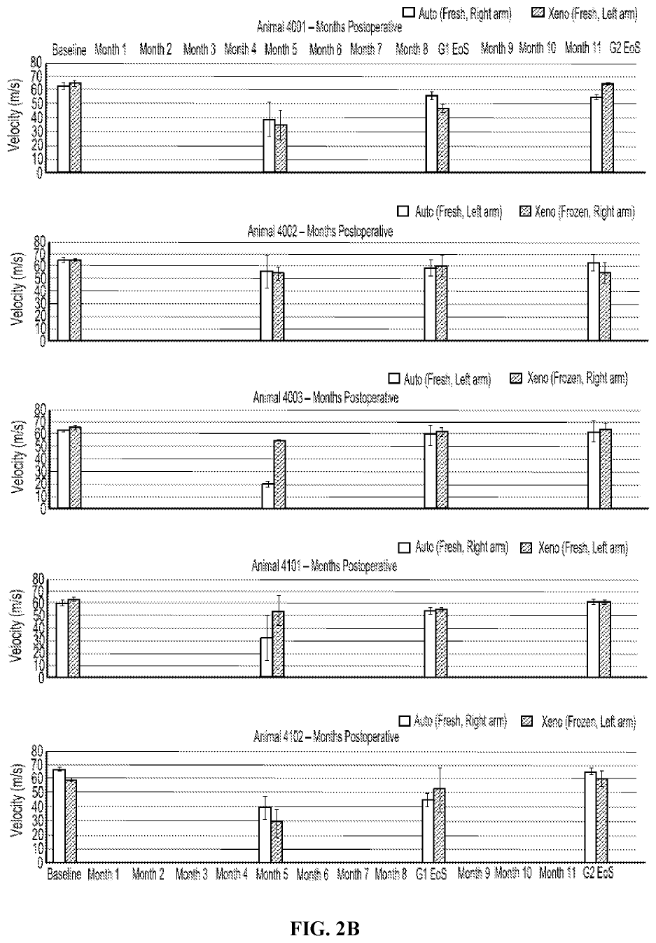

The NCV measured in nerve conduction studies represents the fastest fibers present in the nerve bundle tested. When present, decreased conduction velocity is assumed due to both axonotmesis (axonal loss) and neurapraxia (conduction block). The presence of nerve conduction does not necessarily indicate fully functional muscle innervation. Uneven conduction within the nerve can indicate localized areas of demyelination, remyelination with immature myelin, loss of fibers, or connective tissue blockages. Mean baseline motor NCV for both groups (both arms) was approximately 64 m/s. At the first postgraft analysis (5 months), an overall reduction in NCV in both motor and sensory branches was noted for all animals, down to approximately 40 m/s for motor NCV (43% reduction, Group 1: FIG. 2A; and Group 2: FIG. 2B). At the second post-graft analysis (8.5 months), the motor conduction velocity had further recovered to 55 m/s, or only 15% residual impairment relative to baseline. This indicated that at least a portion of the fast-conducting fibers recovered and remyelinated. At the final assessment (12 months) NCV recovered to 95% of baseline velocity, with no distinguishable difference between either baseline and recovery, or autograft and Xeno-Nerve-grafted arms. Notably, all grafts/animals demonstrated recovery to approximate baseline values for motor NCV by the end of the study.

Taken together, these results demonstrate that nerve xenografts were as effective as autografts in restoring nerve conduction as measured by nerve conduction velocity (NCV) when used for reconstruction of long segmental nerve gaps in a Rhesus monkey model.

### Example 4: Histological Analysis Revealed Healing of the Nerve Defect with Xenograft and Autograft Tissue.

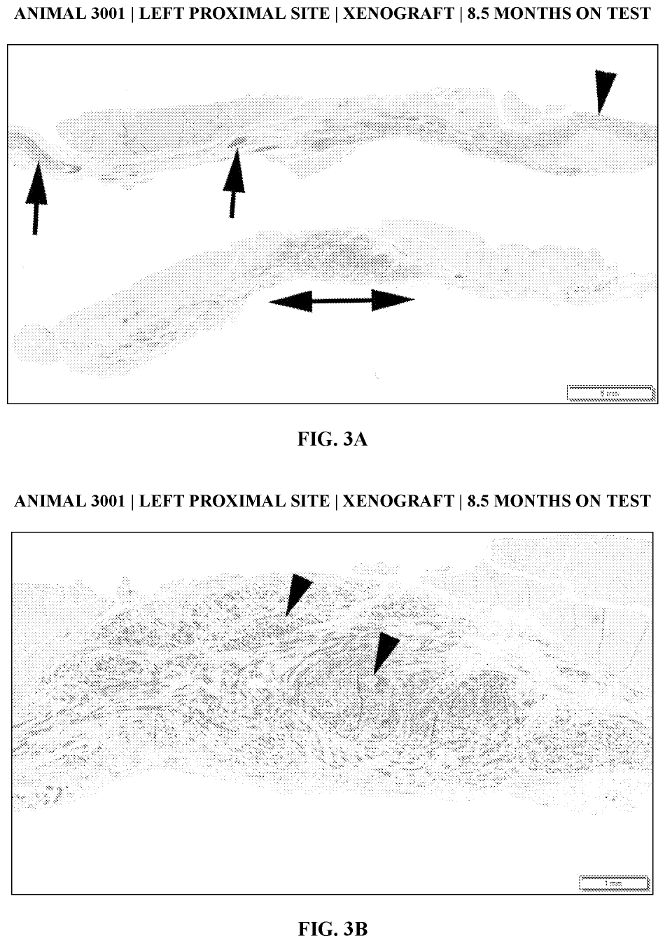

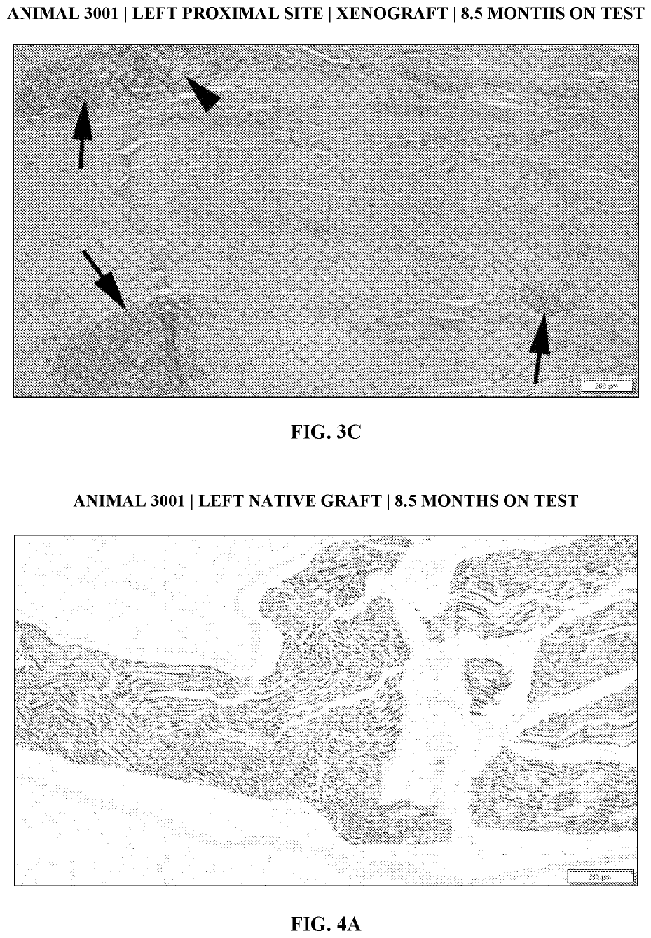

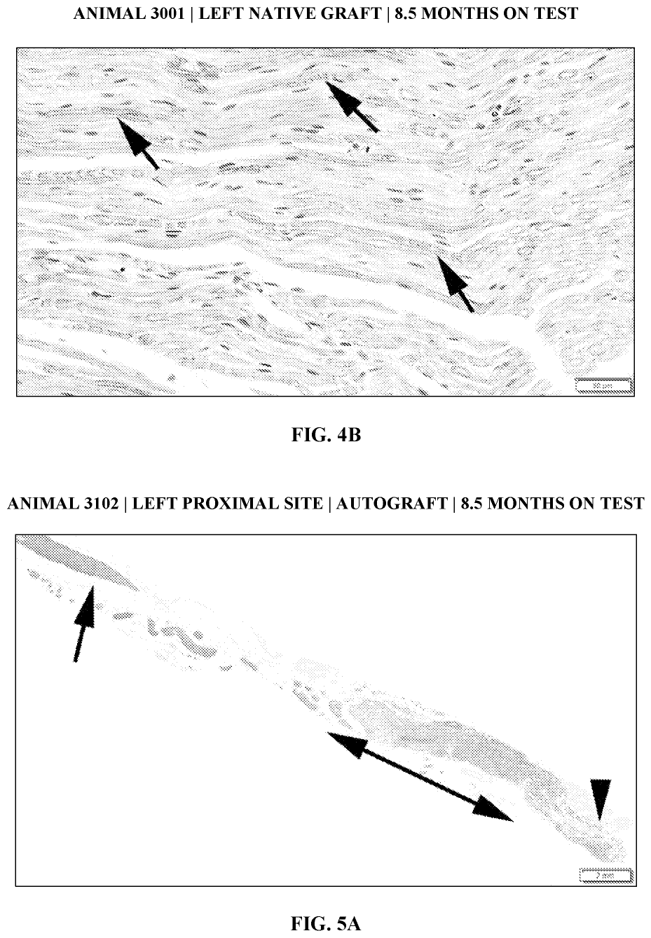

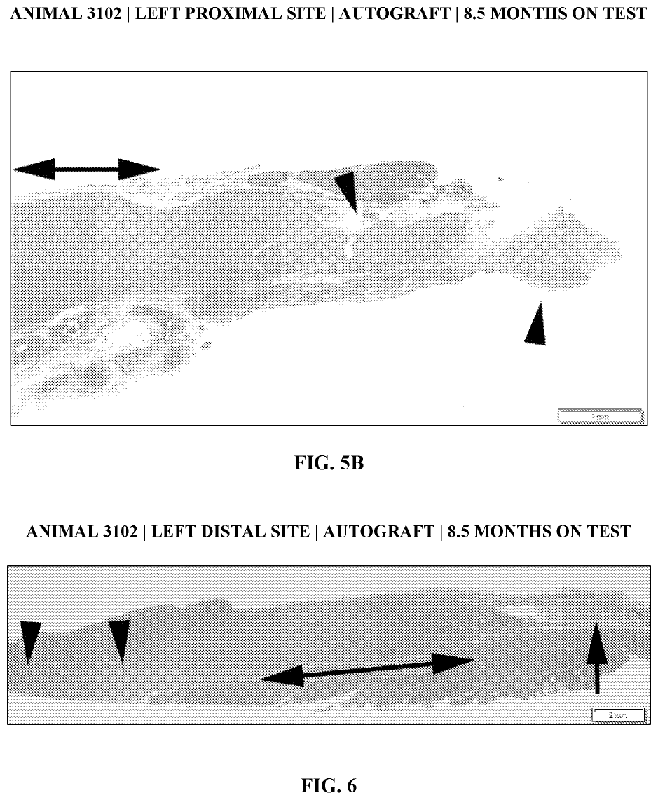

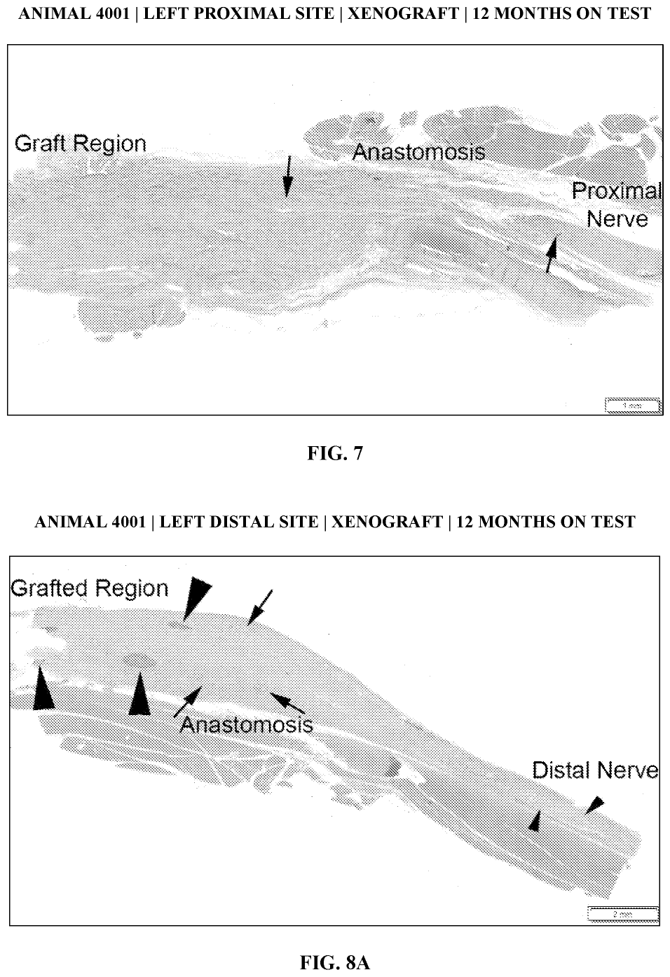

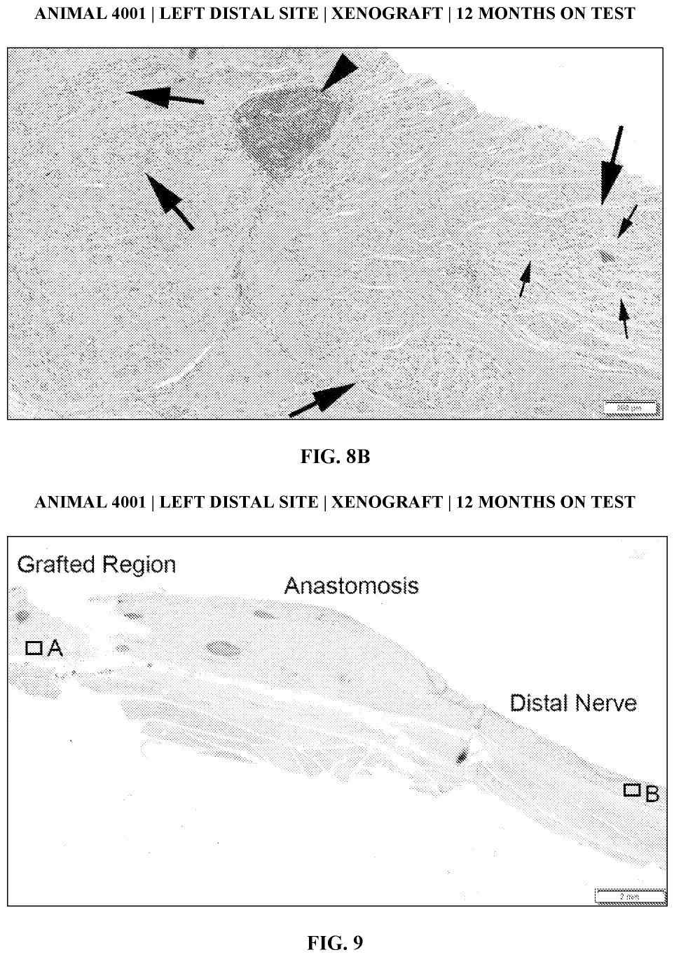

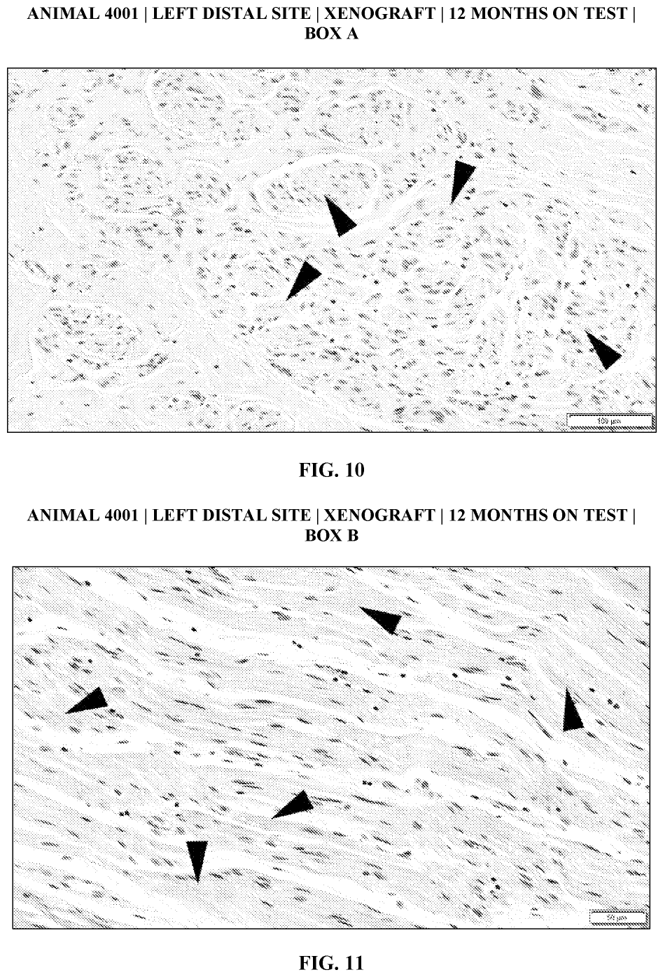

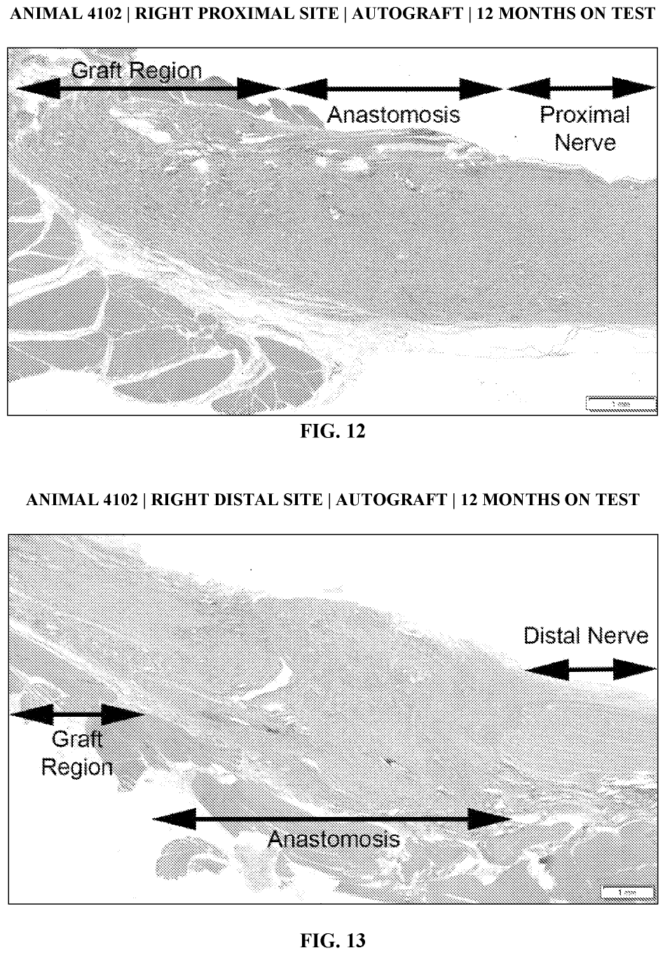

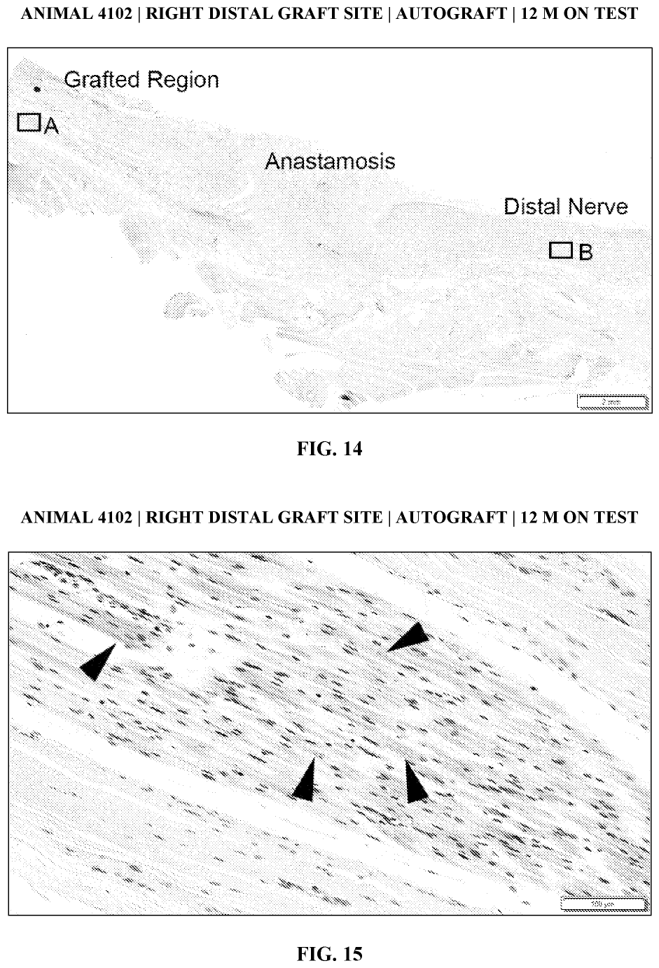

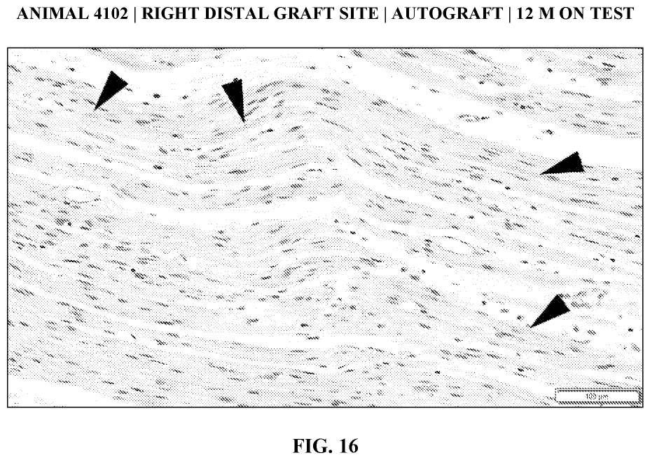

To gain insight into the underlying physiology of the graft, histological analysis of reconstructed nerve was performed. At necropsy, xenograft and autograft nerve segments were collected and fixed in 10% neutral buffered formalin (NBF) and transferred to 70% ethanol (EtOH) after approximately 72 hours. Graft segments and non-target tissues were trimmed, processed, embedded in paraffin, sectioned and stained with hematoxylin and eosin (H&E). Additionally, nerve grafts were stained with Luxol Fast Blue (LFB) and immunohistochemically stained for CD31 (all Group 1 and native graft Group 2 samples only) and NF200 (axonal marker, also known as NF-H). Representative images showing histological analysis of xenografts and autografts are shown in FIGS. 3-16.

### Animal Mortality

No early deaths were observed among animals evaluated in studies described herein.

### Macroscopic Observations

Variable thickening of the graft junctions (proximal and distal) for all nerve graft sites was observed. This thickening correlated microscopically with fibrotic reaction to the anastomosis procedure and variable neuroma formation. These responses were evenly distributed in severity across treatment groups.

### Microscopic Evaluation

### General Comments

While attempts were made to keep the nerve explants as linear as possible, there was a lack of consistent sections parallel to the long axis of the explant, due largely to the variations within the linearity of the explant itself in situ. Specifically, fibrosis during the healing response and fibrous tissue shrinkage led to non-linearity of the graft between the anastomotic sites for many of the explants. Because the mid-section of the nerve had been removed at necropsy, and limited tissue remained for the proximal and distal sections, a cross-section was not taken across the graft.

### Nerve Explant Site Response: Proximal and Distal Anastomoses

The response at the anastomotic sites, both proximal and distal, was characterized by fibrous tissue proliferation, with variable inflammation, generally consisting of foreign body reaction around the sutures. There was variable, multidirectional proliferation of small diameter nerve branches consistent with neuroma formation. There was no clear difference between the autograft and the xenograft in the response at the anastomotic site based upon the type or severity of the response.

### Nerve Explant Site Response: Grafted Region (Graft)

The response across the grafted region demonstrated mild fibrosis with embedded nerve fibers generally coursing longitudinally, or at least in a uniform direction, across the original defect site. Closer to the anastomotic site, the number and irregularity of the fibers increased and was consistent with the neuroma formation as described herein. There was some variation in the size of the nerve fibers in this region. At 8.5 months (time on test), the ‘axon diameter’ scores ranged from ‘2’ (one autograft and one xenograft site) to ‘4’ (one autograft site), with the remainder of scores being ‘3’ (7 out of 10 sites). By the 12-month (time on test) time point, there was a decrease in the overall scores for the xenograft sites (4 out of 5 with score of ‘2’ and 1 out of 5 with score of ‘3’) and an increase in the scores for the sites implanted with autografts (1 out of 5 had a score of ‘3’ and 4 out of 5 had a score of ‘4’). One xenograft site (Animal 4002, right; data not shown) had no clear nerve tissue apparent macroscopically, and the continuity of the graft could not be confirmed microscopically.

There was a notable difference in the infiltration of inflammatory cells within the graft. At the 8.5-month time point, the overall inflammation for the autograft sites was minimal, consisting of scattered lymphocytes and macrophages, with one animal having a lymphoid follicle. Contrastingly, at this same time point, the sites engrafted with xenografts, fresh or frozen, had overall inflammation scores of ‘1’, ‘2’ or ‘3’, due largely to the presences of lymphoid nodules. In animals 3001 (Left), and 3002 (Right), there were infiltrates of lymphocytes and macrophages associated with minimal necrosis of nerve fibers; these two animals also had minimal lymphoid nodules.

At the 12-month time point, within the xenograft implanted sites, there was minimal (1 out of 5 sites) to mild (4 out of 5 sites) overall inflammation, attributable to the presence of lymphoid follicles, along with scattered lymphocytes and macrophages. The autograft implanted sites had minimal numbers of lymphocytes and macrophages scattered through the graft region in 2 out of 5 sites. These findings are consistent with a delayed acquired immune response to the xenograft porcine tissue.

The level of myelination was scored with a ‘demyelination’ score. Higher scores correlate with a lower quantity of myelination present; a score of ‘4’ represents little to no myelination, while a score of 0 represents close to 100% myelination. At 8.5 months, the xenograft implanted graft region had demyelination scores of ‘4’, while the autograft sites had demyelination scores in the graft region of ‘3’ (2 out of 5) and ‘4’ (3 out of 5) suggesting some myelination in several of the autograft sites. At 12 months, demyelination scores for the graft region remained at ‘4’ (4 out of 5) with one score of ‘3’ for the xenograft engrafted sites. The autograft implanted sites demonstrated increasing myelination for 2 out of 5 sites, as evidenced by scores of ‘2’ for those sites. The remaining 3 autograft sites had demyelination score of ‘4’ despite larger nerve diameters, as discussed herein.

### Nerve Explant Site Response: Proximal and Distal Nerve

For consistency and since there was variable amounts of proximal and distal nerve present, evaluation of the proximal and distal nerve was made in the region just beyond where the effects of the anastomotic procedure would impact the nerve. Due to the limitations of the sample preparation, this was chosen on a site-by-site basis.

At the 8.5-month time point, the proximal nerve generally demonstrated minimal to sometimes mild demyelination. Edema, characterized by some separation of nerve bundles and often myxomatous change, was variably present and slightly more severe in the autograft implanted sites, but likely not a significant change. Demyelination was more severe in the distal nerve, but there was no difference between the autograft and xenograft implanted sites. Edema was also evident but not as frequently seen as in the proximal sites.

At the 12-month time point, edema and demyelination was still a feature in the proximal nerve but was minimal and not as frequent as at 8.5 months, with no difference based upon the type of implant. There was minimal inflammation (infiltrates of scattered lymphocytes and histiocytes) in two of the sites implanted with autograft that was considered to be incidental. Within the distal nerve, however, demyelination was still a prominent feature as was seen at 8.5 months, but there was an increased severity for the sites implanted with the xenograft, with equal distribution of severity scores between fresh and frozen. Minimal inflammation (mostly consisting of infiltrates of lymphocytes and histiocytes), was variably observed in the distal nerve; this was not considered to be a significant finding.

### Non-Target Tissue Response

All microscopic findings in the non-target tissues were considered to be incidental, of the nature commonly observed in rhesus monkeys and not considered to be related to treatment with the xenograft test article.

### Summary

Microscopic evaluation of reconstructed segmental nerve gaps in a rhesus monkey model treated with an alpha-1,3-galactosyltransferase knockout swine xenograft and terminated at 8.5 or 12 months demonstrated variable healing of the nerve defect with host (autograft) and xenograft tissue, with somewhat better repair of the nerve defect with the autograft tissue as evidence by larger nerve fibers and increased remyelination.

One xenograft site had no macroscopic or microscopic indication of nerve regeneration across the nerve defect. Inflammation, mostly in the form of lymphoid follicles and infiltrates of lymphocytes and histiocytes, was greater for the xenograft implanted sites, largely reflecting an acquired immune response to the porcine tissue. There was no evidence of a difference between the fresh and frozen xenograft response, however, the number of animals was low precluding adequate comparison in light of the relatively minor variations. There was no evidence of systemic effects of xenograft application.

### OTHER EMBODIMENTS

All of the features disclosed in this specification can be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments can be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases can encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A nerve xenograft for implanting in a human patient, the nerve xenograft comprising nerve tissue derived from a genetically engineered non-human organism.

2. The nerve xenograft of claim 1, wherein the genetically engineered non-human organism has been genetically modified to lack alpha-1,3-galactose epitopes.

3. The nerve xenograft of claim 1 or claim 2, wherein the genetically engineered non-human organism is a swine.

4. The nerve xenograft of any one of claims 1-3, wherein the nerve tissue is derived from the central nervous system of the genetically engineered non-human organism.

5. The nerve xenograft of any one of claims 1-4, wherein the nerve tissue is derived from the peripheral nervous system of the genetically engineered non-human organism.

6. The nerve xenograft of any one of claims 1-5, further comprising cells.

7. The nerve xenograft of claim 6, wherein the cells comprise Schwann cells.

8. The nerve xenograft of claim 6 or claim 7, wherein the cells are endogenous to the genetically engineered non-human organism or wherein the cells are endogenous to the human patient.

9. The nerve xenograft of any one of claims 1-8, further comprising nerve growth factor (NGF).

10. The nerve xenograft of any one of claims 1-9, wherein the genetically engineered non-human organism is an adult, a juvenile, or a fetus.

11. The nerve xenograft of any one of claims 1-10, wherein the nerve xenograft is cryogenically frozen.

12. The nerve xenograft of any one of claims 1-10, wherein the nerve xenograft is fresh.

13. A method of repairing and/or protecting a nerve tissue in a human patient, the method comprising implanting the nerve xenograft of any one of claims 1-12 in a human patient in need thereof.

14. The method of claim 13, wherein the human patient is undergoing surgery that exposes nerve tissue.

15. The method of claim 13 or claim 14, wherein the human patient has a nerve injury.

16. The method of claim 15, wherein the nerve injury comprises a peripheral nerve injury and/or a spinal cord injury.

17. The method of claim 15 or claim 16, wherein the nerve injury is selected from the group consisting of a compressed nerve, a stretched nerve, a crushed nerve, a severed nerve, an inflamed nerve, a demyelinated nerve, and a nerve contusion.

18. The method of claim 17, wherein the nerve injury comprises a severed nerve, and wherein the severed nerve comprises a nerve gap.

19. The method of claim 18, wherein the nerve gap is 3 to 7 centimeters in length.

20. The method of claim 19, wherein the nerve gap is 3.5 to 4.5 centimeters in length.

21. The method of any one of claims 13-20, further comprising administering to the human patient an immunosuppressive agent.

22. The method of claim 21, wherein the immunosuppressive agent is a calcineurin inhibitor (CNI).

23. The method of claim 22, wherein the calcineurin inhibitor is selected from the group consisting of cyclosporine, tacrolimus, and pimecrolimus.

24. The method of claim 25, wherein the calcineurin inhibitor is administered at 0.05 to 5 mg/kg/day.

25. The method of claim 24, wherein the calcineurin inhibitor is administered at 0.1 to 0.25 mg/kg/day.

26. The method of claim 25, wherein the calcineurin inhibitor is administered at 0.15 mg/kg/day.

27. The method of any one of claims 22-26, wherein the immunosuppressive agent is administered prior to implanting the nerve xenograft, concomitantly with implanting of the nerve xenograft, and/or after implanting of the nerve xenograft.

28. The method of any one of claims 21-27, wherein the immunosuppressive agent is administered daily for 3 to 7 months.

29. The method of claim 28, wherein the immunosuppressive agent is administered daily for 6 months.

30. The method of any one of claims 13-29, wherein the nerve xenograft is implanted via surgery.

31. The method of any one of claims 13-30, further comprising contacting the nerve xenograft with one or more biological molecules prior to implanting the nerve xenograft in the human patient.
